(19) United States
(12) Patent Application Publication
(10) Pub. No.: US 2012/0160239 A1
(43) Pub. Date: Jun. 28, 2012

(54) Breathing Apparatus

(75) Inventor: Richard Hingley, Kent (GB)

(73) Assignee: Smiths Medical International Limited, Kent (GB)

(21) Appl. No.: 13/392,705

(22) PCT Filed: Aug. 31, 2010
(86) PCT No.: PCT/GB2010/001639

§ 371 Date: Feb. 27, 2012

(30) Foreign Application Priority Data
Sep. 10, 2009 (GB) 0915819.7

(51) Int. Cl. A61M 16/06; A61M 16/22

(52) U.S. Cl. 128/201.25

(57) ABSTRACT

Breathing apparatus includes a hood enclosing the patient's head and having a gas inlet and outlet located on the hood in the region of the patient's nose and mouth. Inlet tubing connects the gas inlet on the hood with an outlet of a gas supply unit; outlet tubing connects the gas outlet on the hood with an inlet of the gas supply unit. A pump in the gas supply unit draws gas extracted from the outlet tubing through a scrubber to remove carbon dioxide from gas extracted from the enclosure. A mixer valve mixes gas from the scrubber with fresh gas from an oxygen and helium cylinder for supply back to the inlet tubing.

This invention relates to breathing apparatus of the kind including an enclosure adapted to enclose the entire head of a user.

The invention is more particularly concerned with apparatus for providing breathing gas to patients having respiratory problems.

It is known that patients with respiratory problems can benefit from a gas mixture of oxygen and helium, the latter gas reducing the patient's work of breathing. Helium, however, is relatively scarce and expensive so conventional ventilation techniques where exhaled gas is exhausted to atmosphere are very wasteful and costly. The high cost is such that treatment with helium can only usually be provided to patients with severe respiratory problems.

It is an object of the present invention to provide alternative breathing apparatus.

According to one aspect of the present invention there is provided breathing apparatus of the above-specified kind, characterised in that the apparatus includes seal means adapted to provide at least a partial gas seal between the lower end of the enclosure and the neck or shoulder region of the user, gas inlet means opening on the interior of the enclosure in the region of the user's nose or mouth, gas outlet means opening on the interior of the enclosure, inlet and outlet tubing connected with respective inlet and outlet means, gas supply means connected with the inlet and outlet tubing, that the gas supply means includes means arranged to remove at least a part of carbon dioxide in exhaled breath flowing along the outlet tubing, and that the gas supply means is arranged to supply a part at least of the exhaled breath after removal of the carbon dioxide to the inlet tubing.

The gas supply means preferably includes a supply of oxygen. The gas supply means preferably includes a supply of helium. The enclosure is preferably of a flexible plastics material, the gas inlet and outlet means being both supported on the enclosure in the region of the user's nose or mouth. The gas supply means may include a pump arranged to suck gas out of the enclosure and pass it through the means for removing at least a part of the carbon dioxide. The gas supply means may include a sensor responsive to exhalation of the user, the gas supply means being responsive to an output from the sensor indicative of exhalation to inhibit gas supply to the enclosure.

According to another aspect of the present invention there is provided an enclosure for apparatus according to the above one aspect of the present invention.

Breathing apparatus according to the present invention will now be described, by way of example, with reference to the accompanying drawing, in which:

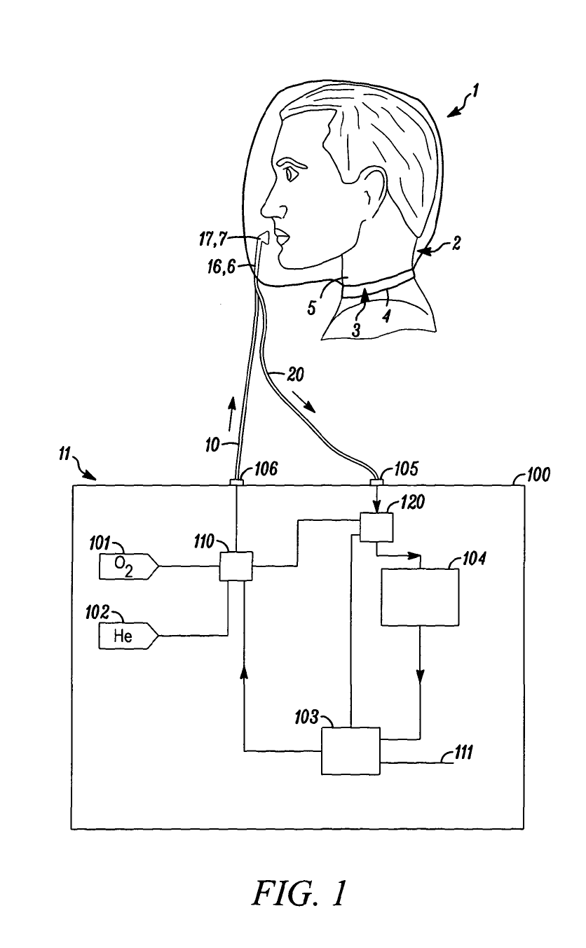

FIG. 1 shows the apparatus schematically with a profile view of the patient;

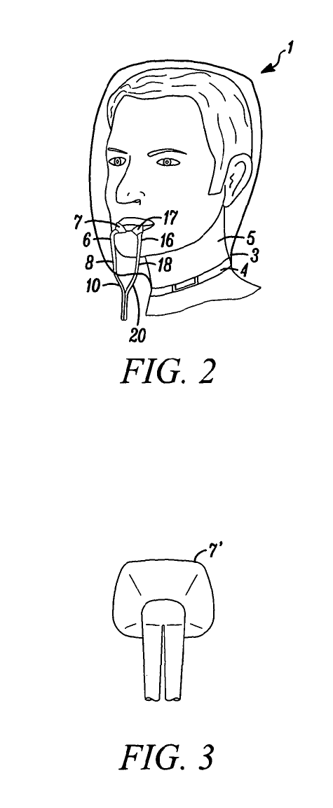

FIG. 2 is a three-quarter view of the enclosure and patient; and

FIG. 3 illustrates an alternative form of gas inlet and outlet.

With reference first to FIGS. 1 and 2, the apparatus includes an enclosure in the form of a hood 1 of a transparent, flexible, impervious plastics material. The size and dimensions of the hood 1 are selected such that it completely encloses the head 2 of the patient, leaving a space between the inside of the hood and the face, which is filled by a mixture of gas supplied to the hood and gas exhaled by the patient. The lower end 3 of the hood 1 has a seal 4 or the like arranged to draw the hood about the neck 5 of the user to provide a partial seal preventing the majority of gas escaping from the hood. The seal 4 could be arranged to tighten the lower end 3 of the hood 1 about the user's neck 5 or it could have an adhesive inner surface that adheres to the user's skin around his neck. It will be appreciated that a range of sizes of hood could be provided for patients of differing sizes. The nature of the hood material is such that it is flexible but self-supporting and will not be inflated or deflated significantly by normal changes in gas pressure within the hood 1. Although the hood 1 is preferably completely transparent, it need not be transparent over the rear of the hood if it were preferable, for example, to make this part of the hood of a different material, perhaps a material that provided greater comfort when the patient was lying down in a supine position.

The hood 1 supports gas inlet and outlet means on the front of the hood by which gas is supplied to and from the hood. The gas inlet means 6 includes a funnel-shape nozzle 7 at the upper end of a tubular support stem 8 mounted inside the hood. The nozzle 7 is directed horizontally towards the mouth of the patient; the stem 8 extends substantially vertically, being supported at its lower end by the wall of the hood 1 through which it extends in the region of the chin. The lower end of the stem 8 joins with flexible tubing 10 outside the hood 1, which extends to a gas supply means or unit 11. The gas outlet means 16 is identical to the inlet means 6, taking the form of a nozzle 17 mounted on a stem 18 alongside the inlet 6 and opening in the region of the mouth. The stem 18 connects with flexible tubing 20, which also extends to the gas supply unit 11.

It will be appreciated that the gas inlet and outlet could be provided in different ways. For example, the inlet and outlets could project through and be supported on the wall of the hood directly opposite the mouth or nose region instead of being supported on the stems 8 and 18 shown in FIGS. 1 and 2. Instead of having two separate funnels 7 and 17, they could be combined in a single funnel 7′, as shown in FIG. 3.

The gas supply unit 11 may be enclosed in a single housing 100 (as shown in FIG. 1) or provided by several discrete components. As shown, the unit 11 includes cylinders 101 and 102 of compressed oxygen and helium although these gases could be provided by external supplies, such as hospital wall gas supplies. The unit 11 also includes a pump 103 or other device that moves gas, such as an entrainment device, and a scrubber 104 or similar device for removing carbon dioxide. The housing 100 includes an inlet 105, which connects with the gas outlet tubing 20, and an outlet 106, which connects with the gas inlet tubing 10. Gas from the oxygen and helium supplies 101 and 102 flows to a mixer valve 110 and from there to the gas outlet 106 and, via the tubing 10 to the inlet nozzle 7 in the hood 1 so that it fills the space within the hood with a mixture of oxygen and helium. Any gas not inhaled by the patient fills the hood 1 and creates a reservoir of oxygen and helium for subsequent breaths or in times when there is an increased tidal volume, such as during periods of increased exertion.

A pressure or flow sensor 120 in the unit 100 detects when the patient exhales. The unit 11 responds to an output of the sensor 120 indicative of exhalation to inhibit gas supply to the hood 1 by switching off gas supply from the mixer valve 110. The unit 11 also turns on the pump 103 to suck gas from the hood 1 with its elevated level of carbon dioxide and draw it through the scrubber 104. The scrubber 104 removes most of the carbon dioxide from the gas mixture and provides a gas output to the pump 103. The pump 103 need only be a low flow pump and may include an additional inlet 111 for air, which is mixed with the gas from the scrubber 104. The pump 103 could be arranged to create a slightly reduced or negative pressure (that is, below atmosphere) within the hood 1 when the patient exhales so that a greater proportion of the used gas within the hood is removed. Because the pressure in the hood is reduced or is less than that outside the hood, there is less escape of gas from the hood. Alternatively, the pump 103 could be arranged to create a slightly positive pressure within the hood 1 so that it reduces the amount of atmospheric air entrained into the hood, but with the disadvantage of increased leakage out of the hood. The pump 103 supplies the scrubbed exhaled gas to the mixer valve 110 with any added fresh air (if used) where it mixes with the oxygen and helium from the cylinders 101 and 102. In this way, the exhaled gas with its elevated helium level is supplied back to the patient so as to conserve a large proportion of the helium. The system also helps recycle some of the unused oxygen, thereby reducing consumption, which may be useful in portable systems.

The hood 1, gas supply unit 11 or associated tubing 10, 20 could include a safety valve (not shown) to prevent asphyxiation by opening to atmosphere if pressure from the gas cylinders 101, 102 or pump 103 should fall below a set level. A safety valve could also be provided to prevent excess pressure, opening to vent gas in the hood to atmosphere if pressure should rise above a different set level.

1-7. (canceled)

8. Breathing apparatus including an enclosure adapted to enclose the entire head of a user and including a seal adapted to provide at least a partial gas seal between the lower end of the enclosure and the neck or shoulder region of the user, characterized in that the enclosure supports a gas inlet opening on the interior of the enclosure in the region of the user's nose or mouth, that the apparatus includes a gas outlet opening on the interior of the enclosure, inlet and outlet tubing connected with respective ones of the inlet and outlet and a gas supply connected with the inlet and outlet tubing, that the gas supply includes a supply of helium, that the gas supply is arranged to remove at least a part of carbon dioxide in exhaled breath flowing along the outlet tubing, and that the gas supply is arranged to supply a part at least of the exhaled breath to the inlet tubing after removal of the carbon dioxide.

9. Breathing apparatus according to claim 8, characterized in that the gas supply includes a supply of oxygen.

10. Breathing apparatus according to claim 8, characterized in that the enclosure is of a flexible plastics material.

11. Breathing apparatus according to claim 8, characterized in that the gas supply includes a pump arranged to suck gas out of the enclosure and to pass it through a scrubber for removing at least a part of the carbon dioxide.

12. Breathing apparatus according to claim 8, characterized in that the gas supply includes a sensor responsive to exhalation of the user, and that the gas supply is responsive to an output from the sensor indicative of exhalation to inhibit gas supply to the enclosure.

13. An enclosure for apparatus according to claim 8.
